(19) United States
(12) Patent Application Publication
(10) Pub. No.: US 2019/0247422 A1
(43) Pub. Date: Aug. 15, 2019

(54) COMPOSITION FOR PROMOTING SECRETORY IGA SECRETION

(71) Applicant: KANEHIDE BIO CO.,LTD., Itoman-shi (JP)

(72) Inventors: Mikio MIYAGI, Itoman-shi (JP); Keiichiro INAFUKU, Itoman-shi (JP); Yukari IKEHARA, Itoman-shi (JP)

(73) Assignee: KANEHIDE BIO CO.,LTD., Itoman-shi (JP)

(21) Appl. No.: 16/343,763

(22) PCT Filed: Oct. 31, 2017
(86) PCT No.: PCT/JP2017/039209

§ 371 Date: Apr. 21, 2019

(30) Foreign Application Priority Data
Nov. 1, 2016 (JP) 2016-214594

(51) Int. Cl. A61K 31/737; A61P 37/04

(52) U.S. Cl. CPC A61K 31/737; A61P 37/04

(57) ABSTRACT

It is an objective of the present invention to provide a composition that is sufficiently safe to allow daily ingestion and that has an ability to promote the secretion of s-IgA into saliva in a continuous and age-independent manner. The above objective is achieved by the composition for promoting secretion of secretory IgA, comprising as an active ingredient Okinawa mozuku-derived fucoidan with a recommended daily intake of 1,000 mg or more. The composition has the mucosal immunostimulatory effect, anti-infection effect or anti-allergy effect and is expected to be used for ameliorating, mitigating, suppressing, treating or preventing diseases or symptoms on which these effects are expected to work, such as bacterial infection, viral infection, protozoan infection, food allergy, pollenosis, gastritis, enteritis, diarrhea, ulcerative colitis, rhinitis, bronchitis, bronchial asthma, Löffler's syndrome and stomatitis, as well as any symptoms resulting therefrom. The composition is also expected to be used in immunotherapies and induction of immunotolerance.

### CROSS-REFERENCE TO RELATED APPLICATION

The present application claims the benefit of priority to Japanese Patent Application No. 2016-214594 filed on Nov. 1, 2016, the disclosure of which is incorporated herein by reference in its entirety.

### TECHNICAL FIELD

The present invention relates to compositions for promoting secretion of secretory IgA.

### BACKGROUND ART

Secretory IgA (s-IgA) is an immunoglobulin that plays a key role in mucosal immunity. It is a dimeric IgA consisting of two monomeric serum IgA molecules joined via a polypeptide called J chain with a glycoprotein secretory component bound to the dimer. Unlike monomeric IgA, s-IgA with the bound secretory component is less susceptible to proteases even when it is present in an exocrine secretion. Also, IgA is classified in two subclasses. IgA1 among the two subclasses is predominant in the upper respiratory tract, whereas IgA2 among the two subclasses is predominant in the gastrointestinal tract (refer to Non-Patent Document 1 listed below; the disclosure of the document is incorporated herein by reference in its entirety).

Since an IgA has two antigen-binding sites, an s-IgA has a total of four antigen-binding sites. S-IgA with high antigen-recognition ability is secreted from plasma cells of mucosal tissue and is primarily present in saliva, tear, colostrum, nasal mucosa, mother's milk, trachea, bronchus, intestinal tract and other exocrine secretions where it plays a crucial role in mucosal immunity. Specifically, s-IgA has neutralizing and aggregation activities and is involved in homeostasis and body defense on mucosal surface.

For example, microorganisms and allergens entering the upper respiratory tract are caused to aggregate by the action of s-IgA present in exocrine secretions before invading the living body. They are then made harmless without being destroyed, for example, by being transported into the stomach by the ciliary transport of the upper respiratory tract mucosa. By this mechanism, the mucosal surface remains less susceptible to inflammation under the action of s-IgA. That is, s-IgA acts to inhibit adherence of microorganisms and allergens to the mucosa; for example, in the oral and nasal cavity, s-IgA secreted in saliva can serve as an immune barrier against microorganisms and viruses infecting the upper respiratory tract. Specifically, it is believed that newborns passively acquire immunity against gastrointestinal infection by ingesting s-IgA in mother's milk.

Fucoidan is a type of viscous water-soluble polysaccharide found in various species of brown algae, including mozuku (Cladosiphon okamuranus), kombu (Laminariaceae and Undaria) and wakame (pinnatifida). Its major constituent sugar is fucose, which is characteristically modified with a sulfate group. Fucoidan is classified into various types by its molecular weight, constituent sugars and the manner of sulfate linkage. The difference between various types of fucoidan is mainly attributed to the types of organisms from which they are derived.

Non-Patent Document 2 listed below, the entire disclosure of which is incorporated herein by reference, states that s-IgA secretion in saliva was promoted by continuous daily intake of 1,000 mg/day of a food product formulated with Okinawa mozuku-derived fucoidan, mekabu-derived fucoidan and an Agaricus mycelium extract powder.

Prior Art List

### Non-Patent Document

Non-Patent Document 1: Yuichi Kurono, Japan Society of Immunology and Allergology in Otolaryngology, 2004, 22(1), pp. 1-5

Non-Patent Document 2: Yoshiyuki Miyazaki et al., Food Style 21, 2014, vol. 18, no. 12, pp. 21-25

### SUMMARY OF THE INVENTION

### Technical Problem

According to Non-Patent Document 2, the rate of s-IgA secretion into saliva has increased at week 4 or week 12 of ingestion of the food product described in Non-Patent Document 2. However, the rate of s-IgA secretion into saliva has decreased at week 8 of the ingestion of the food product rather than before ingestion of the food product. Thus, the ability of the food product described in Non-Patent Document 2 to increase the rate of s-IgA secretion into saliva is discontinuous, rather than continuous.

Further according to Non-Patent Document 2, no increase in the rate of s-IgA secretion into saliva was observed in subjects aged 40 or more at any of the time points during ingestion of the food product described in Non-Patent Document 2. Rather, at week 8 of ingestion, the rate of s-IgA secretion into saliva has decreased as compared to prior to ingestion. The ability of the food product described in Non-Patent Document 2 to increase the rate of s-IgA secretion into saliva is therefore dependent on the age of subjects.

On the other hand, no food or drink products have been known thus far that can increase the rate of s-IgA secretion into saliva in a continuous and age-independent manner and that is safe to ingest on a daily basis.

Accordingly, it is an object of the present invention to provide a composition that is sufficiently safe to allow daily ingestion and that has an ability to promote the secretion of s-IgA into saliva in a continuous and age-independent manner.

### Solution to Problem

In an effort to find solutions to the above-identified problems, the present inventors have examined various traditionally consumed food materials that can potentially increase the rate of s-IgA secretion into saliva. Consequently, the present inventors have surprisingly found that the rate of s-IgA secretion into saliva can be significantly enhanced through a predetermined daily intake of Okinawa mozuku-derived fucoidan. Further surprisingly, such an ability to promote secretion of s-IgA into saliva was continuously exhibited from week 4 through week 12 in individuals who ingested the fucoidan irrespective of their age. Also, since Okinawa mozuku-derived fucoidan is a traditionally eaten food product, its safety has already been confirmed. Based on these findings, the present inventors have succeeded in producing a composition for promoting secretion of secretory IgA that contains as an active ingredient Okinawa mozuku-derived fucoidan with a predetermined recommended daily intake. It is these findings and successful examples that have ultimately led to completion of the present invention.

Accordingly, aspects of the present invention provide the following:

[1] a composition for promoting secretion of secretory IgA, comprising as an active ingredient Okinawa mozuku-derived fucoidan with a recommended daily intake of 1,000 mg or more;
[2] the composition according to [1], wherein the secretory IgA is secretory IgA in saliva.
[3] a mucosal immunostimulatory composition, an anti-infection composition or an anti-allergy composition, comprising as an active ingredient Okinawa mozuku-derived fucoidan with a recommended daily intake of 1,000 mg or more;
[4] the composition according to any one of [1] to [3], wherein the composition is for ingestion by an individual having a rate of s-IgA secretion into saliva of 150 μg/min or less.
[5] the composition according to any one of [1] to [4], wherein the composition is free of a component selected from the group consisting of mekabu-derived fucoidan and Agaricus mycelium extract powder;
[6] a method for treating or preventing infection or allergy, comprising administering Okinawa mozuku-derived fucoidan with a recommended daily intake of 1,000 mg or more to an individual;
[7] the method according to [6], wherein the individual has a rate of s-IgA secretion into saliva of 150 μg/min or less; and
[8] the method according to [6], wherein the individual has a disease or symptom selected from the group consisting of bacterial infection, viral infection, protozoan infection, food allergy, pollenosis, gastritis, enteritis, diarrhea, ulcerative colitis, rhinitis, bronchitis, bronchial asthma, Löffler's syndrome, and stomatitis.

### Advantageous Effects of Invention

Through its ability to promote secretion of secretory IgA by active ingredients, in particular its ability to promote secretion of secretory IgA in saliva or mucosal tissue, the composition according to one aspect of the present invention exhibits mucosal immunostimulatory effect, anti-infection effect and anti-allergy effect and is expected to be used for ameliorating, mitigating, suppressing, treating or preventing diseases or symptoms on which these effects are expected to work, such as bacterial infection, viral infection, protozoan infection, food allergy, pollenosis, gastritis, enteritis, diarrhea, ulcerative colitis, rhinitis, bronchitis, bronchial asthma, Löffler's syndrome and stomatitis, as well as any symptoms resulting therefrom. The composition is also expected to be used in immunotherapies and induction of immunotolerance.

### BRIEF DESCRIPTION OF DRAWINGS

FIG. 1 is a graph showing changes in the rate of s-IgA secretion into saliva, as described in Examples below. “**” in the figure indicates that p<0.01.

FIG. 2 is a graph showing changes in the concentration of s-IgA in saliva, as described in Examples below. “**” in the figure indicates that p<0.01.

FIG. 3 is a graph showing changes in the rate of saliva secretion, as described in Examples below.

### MODES FOR CARRYING OUT THE INVENTION

While secretory IgA secretion-promoting compositions, as well as mucosal immunostimulatory compositions, anti-infection compositions and anti-allergen compositions according to one aspect of the present invention (which may be collectively referred to as “composition according to one aspect of the present invention,” hereinafter) will now be described in further details, the scope of the present invention is not limited to what is described in this section; rather, the present invention may take various other forms to the extent that its objectives are achieved.

Unless otherwise specified, each term used in the present specification is used in the meaning commonly used by those skilled in the art and should not be construed to have any meaning that is unduly limiting.

For example, “composition” as used herein has a commonly used meaning that is not particularly limiting; for example, the term includes combinations of two or more materials, specifically, combinations of an active ingredient with one or more materials, combinations of two or more active ingredients, and more specifically, solid compositions and liquid compositions containing one or more active ingredients in combination with one or more solids or solvents.

The composition according to one aspect of the present invention contains at least Okinawa mozuku-derived fucoidan as an active ingredient that gives the composition an ability to promote secretion of secretory IgA.

The secretory IgA (s-IgA) is not particularly limited and may be any commonly known dimeric IgA, including for example dimeric IgA found in saliva, tear, colostrum, nasal mucosa, mother's milk, trachea, bronchus, intestinal tract and other exocrine secretions.

Okinawa mozuku-derived fucoidan may be of any structure or molecular weight that is not particularly limited as long as it is derived from Cladosiphon okamuranus Tokida, a mozuku seaweed grown in Okinawa prefecture.

Okinawa mozuku-derived fucoidan may be produced by any process that is not particularly limited as long as such process can extract polysaccharides from plant bodies in commonly known manners. A non-limiting specific exemplary process for producing Okinawa mozuku-derived fucoidan is described in Japanese Patent No. 3,408,180 B and is described below.

Frozen Okinawa mozuku is thawed and cut into several millimeter pieces. The wet Okinawa mozuku pieces are transferred to an extraction vessel. Water is added to the vessel and heated. 0.1 to 10% of an organic acid such as citric acid with respect to the weight of mozuku is added for extraction. Subsequently, the extract is neutralized and centrifuged, and is then subjected to ultrafiltration to desalt the product. The resulting product is then sterilized and concentrated to obtain high purity fucoidan as a citric acid extract of Okinawa mozuku.

The ability to promote secretion of secretory IgA refers to any ability to increase the amount of endogenous s-IgA present in a living body and its mechanism is not particularly limited. For example, the ability includes the ability to promote secretion of s-IgA from the plasma cells of mucosal tissue. Specific aspects of the ability to promote secretion of secretory IgA may be any one of the abilities selected from the group consisting of the abilities to increase the rate of secretion and the concentration of s-IgA in a living body, the abilities to maintain the increased rate of secretion and concentration of s-IgA, and the abilities to suppress decrease in the increased rate of secretion and concentration of s-IgA.

As described in Examples below, the Okinawa mozuku-derived fucoidan that serves as an active ingredient exhibits an ability to promote secretion of s-IgA into saliva of individuals who ingest the fucoidan in a continuous and age-independent manner. It is known that in the oral and nasal cavities, s-IgA secreted into saliva can make pathogens and allergens invading the upper respiratory tract harmless. It is therefore preferred that the composition according to one aspect of the present invention be a composition that has the ability to promote secretion of secretory IgA into saliva.

The ability to promote secretion of secretory IgA may be evaluated by any method that is not particularly limited; for example, it can be evaluated by measuring the rate of secretion or the concentration of s-IgA. For example, methods comprising measuring the rate of secretion of s-IgA into saliva or the concentration of s-IgA in saliva include, but are not limited to, the method comprising the following steps: sampling saliva of an individual who ingests the composition using a commercially available kit while maintaining normal oral or gastrointestinal environment of the individual; then immunoenzymologically measuring the secretion rate and the concentration of s-IgA for the saliva sample using a commercially available kit; then comparing the measured values with value measured before ingestion of the composition according to one aspect of the present invention; and determining whether or not the secretion of secretory IgA has been enhanced.

The degree of the effect of promoting secretion of secretory IgA is not particularly limited; for example, the degree of the effect may be such that an increase in the rate of secretion or the concentration of s-IgA as compared to when the composition according to one aspect of the present invention is not ingested is observed at week 4, week 8 or week 12 of ingestion. Preferably, the degree of the effect is such that an increase in the rate of secretion or the concentration of s-IgA is observed at week 4 and week 8 or at week 8 and week 12 of ingestion. More preferably, the degree of the effect is such that an increase in the rate of secretion or the concentration of s-IgA is observed at week 4, week 8 and week 12 of ingestion. Even more preferably, the degree of the effect is such that the rate of secretion or the concentration of s-IgA is increased at week 8 and week 12 as compared to week 4.

In addition, the degree of the effect of promoting secretion of secretory IgA of the composition according to one aspect of the present invention is preferably such that an increase in the rate of secretion or the concentration of s-IgA in an individual who ingests the composition according to one aspect of the present invention is observed independent of the age of the individual as compared to when the composition is not ingested. More preferably, the degree of the effect of promoting secretion of secretory IgA of the composition of the present invention is such that an increase in the rate of secretion or concentration of s-IgA in an individual aged 40 or more who ingest the composition according to one aspect of the present invention is observed as compared to when the composition is not ingested.

In order for the composition according to one aspect of the present invention to exhibit the ability to promote secretion of secretory IgA, the recommended daily intake of Okinawa mozuku-derived fucoidan as an active ingredient is 1,000 mg or more. Specifically, the intake of the composition according to one aspect of the present invention depends on the amount of Okinawa mozuku-derived fucoidan as an active ingredient present in the composition.

While the recommended daily intake of Okinawa mozuku-derived fucoidan is not subject to any particular limitations so long as it is any amount equal to or greater than 1,000 mg, it is for example in the range of 1,000 to 2,000 mg, preferably in the range of 1,100 to 1,500 mg, and more preferably about 1,175 mg. It should be noted that the recommended daily intake is the amount intended for ingestion by adults and may be lower than the specified amounts for ingestion by those who are younger than adult age or by those who are in poor health conditions. The number of times the composition according to one aspect of the present invention can be ingested is not particularly limited; for example, it can be ingested once to three times a day. The composition may also be ingested less or more times a day depending on the recommended daily intake of Okinawa mozuku-derived fucoidan as an active ingredient.

The composition according to one aspect of the present invention may take various forms including a composition for food or drink products, a composition for pharmaceutical products or other suitable forms depending on the components present in the composition. Accordingly, one specific aspect of the present invention is a composition for food or drink products, a composition for pharmaceutical products, a composition for quasi-drugs, a composition for cosmetic products, a composition for animal feeds, or the like which contains Okinawa mozuku-derived fucoidan as an active ingredient with the recommended daily intake of 1,000 mg or more.

In addition to Okinawa mozuku-derived fucoidan, the composition according to one aspect of the present invention may contain other components. Other components are not particularly limited; examples may include excipients, extenders, binders, thickeners, preservatives, isotonizing agents, buffers, pH conditioners, suspending agents, stabilizers, emulsifiers, sugar sweeteners, starches, modified starches, degraded starches, salts, flavors, colors, acidulants, flavor raw materials, nutrients, seasonings, and animal and plant materials, such as fruit juices and eggs. The amounts of other components are not particularly limited as long as they do not interfere with objectives of the present invention.

It should be noted that Non-Patent Document 2 states that a composition containing mekabu-derived fucoidan and Agaricus mycelium extract powder in addition to Okinawa mozuku-derived fucoidan may not have the ability to promote secretion of s-IgA into saliva in a continuous and age-independent manner. Accordingly, the composition according to one aspect of the present invention preferably does not contain mekabu-derived fucoidan or Agaricus mycelium extract powder and more preferably, the composition contains neither mekabu-derived fucoidan nor Agaricus mycelium extract powder.

The composition according to one aspect of the present invention may be provided in any commonly used form that is not particularly limited. For example, when the composition is provided in the form of a composition for food or drink products, it may take any form such as solid, liquid, gel, suspension, cream, sheet, stick, powder, particles, granules, tablets, bar, plate, block, paste, capsules and caplets. When the composition is provided in the form of a composition for pharmaceutical products, it may take the form of for example injection (intramuscular, subcutaneous, intradermal), oral preparation or nasal preparation. For example, the composition for pharmaceutical products may be prepared as a vaccine for treating allergic diseases such as food allergies. When the composition for pharmaceutical products is provided in the form of vaccine, it may be prepared as a mixed cocktail vaccine containing different active ingredients.

One specific aspect of the composition for food and drink products is, for example, a functional food and drink product that provide certain functionalities to living bodies. Examples of functional food and drink products include so-called health food and drink products such as Food and Drink for Specified Health Uses, Food and Drink with Functional Claims, Food and Drink with Nutrient Function Claims, Food and Drink with Health Claims, Food and Drink for Special Dietary Uses, nutritional supplementary food and drink, health supplementary food and drink, supplements, and food and drink for beauty, as well as food and drink products for specific type of people, such as food and drink for infants, food and drink for expectant and nursing mothers, and food and drink for aged person. Food and Drink with Functional Claims further includes health food and drink products having health claims based on the food product standards specified by CODEX (FAO/WHO collaborative committee for food standards).

The composition according to one aspect of the present invention may be packaged in any suitable form that is not particularly limited and that is appropriately selected depending on the form in which the composition is applied; examples of such packages include blister packs, such as PTPs; strip packages; heat seals; aluminum pouches; film packages using plastics and synthetic resins; glass containers, such as vials; and plastics containers, such as ampoules.

Another aspect of the present invention is a mucosal immunostimulatory composition, an anti-infection composition and an anti-allergy composition, containing as an active ingredient Okinawa mozuku-derived fucoidan with a recommended daily intake of 1,000 mg or more.

Without being bound by any theory or presumption, it is believed that foreign matters, such as microorganisms and allergens, generally bind to s-IgA and are transported and ultimately made harmless. As a result, the immune system of mucosal tissue is kept healthy. Thus, Okinawa mozuku-derived fucoidan can exhibit its mucosal immunostimulatory effect, anti-infection effect and anti-allergy effect through promotion of secretion of s-IgA.

The mucosal immunostimulatory composition may be any commonly known composition of such kind that is not particularly limited; for example, it may be a composition having a mucosal immunostimulatory effect that can enhance protection against pathogens, allergens and other foreign substances by activating immune functions of the mucosal tissue in individuals who have ingested the composition.

The anti-infection composition and the anti-allergy composition may each be any commonly known composition of such kind that is not particularly limited; for example, they may be a composition intended for ameliorating, mitigating, suppressing, treating or preventing symptoms of infections or allergies, as well as symptoms of diseases associated with infections or allergies and symptoms of autoimmunity, or a composition intended for use in immunotherapies.

The anti-infection composition and the anti-allergy composition are each preferably a composition directed to infections and allergies mediated by the mucosal tissue in the gastrointestinal system, including oral cavity, pharynx, nasal cavity, stomach, bowel, and other organs. More preferably, the anti-infection composition and the anti-allergy composition are each a composition directed to bacterial infection, viral infection, protozoan infection, food allergy, pollenosis, gastritis, enteritis, diarrhea, ulcerative colitis, rhinitis, bronchitis, bronchial asthma, Löffler's syndrome, stomatitis, and other diseases. In view of the fact that the Okinawa mozuku-derived fucoidan, the active ingredient, can promote secretion of s-IgA into saliva, it is even more preferred that the anti-infection composition and the anti-allergy composition be directed to infections and allergies mediated by the mucosal tissue of the oral cavity, pharynx and nasal cavity.

The mucosal immunostimulatory effect, the anti-infection effect and the anti-allergy effect may be evaluated by any method known by those skilled in the art that is not particularly limited; specifically, these effects may be evaluated by determining the degree of symptoms of infections and allergies in individuals who have ingested the composition, by determining the concentration of pathogens and allergens in the individuals, or by determining the types of immune cells and their respective proportions in the individuals.

The composition according to one aspect of the present invention may be ingested not only by humans, but also by other animals including mammals, such as dogs, cats, cows, and horses. The subject to which the present composition can be applied is not limited to healthy individuals, but preferably also includes individuals having a low concentration or a low secretion rate of s-IgA. Individuals in which the rate of s-IgA secretion into saliva is 150 μg/min or less are more preferred. Those having compromised immune functions or for whom treatment or prevention of infections or allergies is desired are even more preferred.

The composition according to one aspect of the present invention may be administered by any suitable manner that is not particularly limited; for example, the composition may be administered via oral or parenteral route. Examples of parenteral route include, but are not limited to, absorption through the mucosa of oral cavity, nasal cavity, or pharynx.

When ingested orally, the composition according to one aspect of the present invention may induce or enhance oral immune tolerance. The resulting oral immune tolerance can help ameliorate, mitigate, suppress, treat or prevent symptoms of allergies such as food allergies, as well as symptoms of autoimmunity.

Specific embodiments of the composition according to one aspect of the present invention include, but are not limited to, hard capsules, solutions, granules, and tablets containing Okinawa mozuku-derived fucoidan and an excipient selected from the group consisting of hydroxypropyl methylcellulose (HPMC), xanthan gum, pullulan, crystalline cellulose, sucrose fatty acid ester, glycerol fatty acid ester, calcium phosphate, and gelatin.

The present invention will now be described in further detail with reference to the following Examples, which are not intended to limit the present invention. The present invention may take various forms to the extent that the objectives of the present invention are achieved.

### EXAMPLES

In order to study the immunostimulatory effect provided by Okinawa mozuku-derived fucoidan, the rate of s-IgA secretion into saliva, the concentration of s-IgA in saliva, and the rate of saliva secretion after ingestion of a food product containing Okinawa mozuku-derived fucoidan were evaluated using the procedures described below.

### 1. Test Food Product

A food product manufactured by Kanehide Bio Co. under a trade name “Mozuku Extract Powder”™ was used. The product was provided in the form of hard capsules and contained a citric acid extract of Okinawa mozuku (Cladosiphon okamuranus Tokida) grown in Okinawa prefecture (the major component of which was fucoidan), and hydroxypropyl methylcellulose (HPMC). The daily intake of Test Food Product was 1,175 mg based on Okinawa mozuku-derived fucoidan.

### 2. Test Samples

Saliva samples were collected from subjects by the following procedure.

The subjects were instructed to brush their teeth by brushing without any polishing agent or rinsing agent and to then rinse their mouths and gargle with approximately 150 ml of water. The subjects rinsed their mouths twice with their mouths closed and gargled twice with their mouths open.

Following rinsing/gargling, the subjects were left to sit still for 15 minutes. For collection of saliva, a Salisoft™ saliva collection tube manufactured by Assist Co. was used. The tare weight of Salisoft was measured and the subjects were instructed to place a sponge that comes with Salisoft in their mouths for 120 seconds to allow saliva to soak into the sponge. The saliva-soaked sponge was then placed back into Salisoft and the weight of Salisoft with saliva was measured. Subsequently, Salisoft was centrifuged at 2,000 rpm for 5 minutes at room temperature and the collected saliva (approx. 1 g) was dispensed into microtubes and frozen to serve as test samples. Saliva samples were collected during the morning in such a manner that collection times are within a two-hour range on each of the test days.

### 3. Measurement Method

The concentration of s-IgA in saliva was determined by using “EIAs-IgA Test” manufactured by Medical & Biological Laboratories Co. according to the manufacturer's instruction.

### 4. Test Method

Of males and females aged 20 to less than 65 years, 11 males and 11 females determined in the pretest to have a relatively low rate of s-IgA secretion into saliva were selected.

Subjects were asked to ingest Test Food Product once daily, 5 capsules each time (equivalent to 1,175 mg of Okinawa mozuku-derived fucoidan), with a glass of cold or warm water (approx. 150 ml). Test Food Product was given over a period of 12 weeks (84 days).

Saliva samples were collected from subjects and the concentration of s-IgA in saliva and the rate of saliva secretion were determined on Week 4 (Day 29), Week 8 (Day 57) and Week 12 (Day 85) from the start of the ingestion period. Subjects were asked to refrain from alcohol or medication on the day before the test and asked to finish meals or fluid intake by around 10 o'clock in the evening and to go to bed by around 12 o'clock to ensure enough sleep. Subjects were further instructed to finish brushing teeth by one hour before the test and were subjected to the test under a fasted condition. On the test day, smoking was not allowed until the end of the test.

The changes in the measured values from before the ingestion period were evaluated using One-Sample t-test.

### 5. Test Results

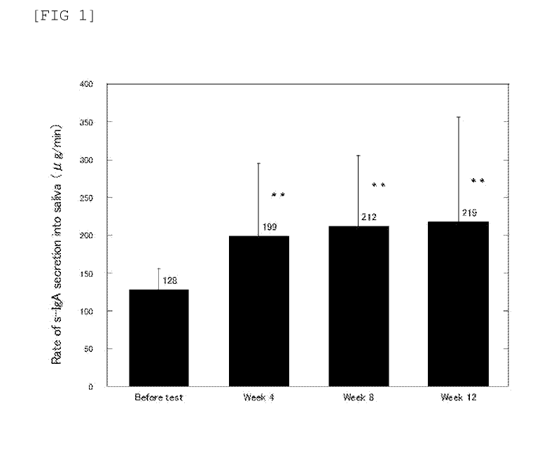

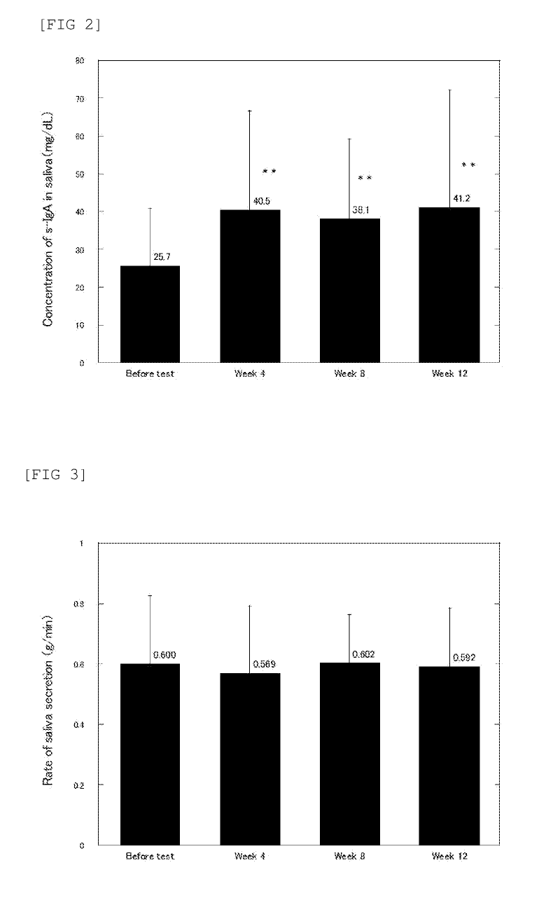

FIGS. 1 to 3 depict changes over time in the rate of s-IgA secretion into saliva, the concentration of s-IgA in saliva, and the rate of saliva secretion, respectively. As can be seen from the figures, significant differences were observed in the rate of s-IgA secretion into saliva and the concentration of s-IgA in saliva as compared with the corresponding values obtained in the pretest (p<0.01). On the other hand, no significant difference was observed in the rate of saliva secretion. Subjects were divided into those aged 40 or more (13) and those aged less than 40 (9), and Two-Sample t-test was conducted with respect to the two groups. The results indicated no significant differences between the two groups.

These results indicate that Okinawa mozuku-derived fucoidan when ingested can promote secretion of s-IgA into saliva. Okinawa mozuku-derived fucoidan is particularly effective in promoting s-IgA secretion into saliva in an age-independent manner in individuals with less s-IgA secreted in their saliva, for example, those who have compromised immune system and are therefore incapable of, or have a possibility to become incapable of properly eliminating pathogens and allergens via their oral and nasal cavities.

### INDUSTRIAL APPLICABILITY

Due to its ability to promote secretion of secretory IgA, the composition according to one aspect of the present invention is useful not only for healthy individuals, but also for those who ingest the composition with an expectation of ameliorating, mitigating, suppressing, treating, or preventing diseases or symptoms caused by decreased secretion of secretory IgA. The composition of the present invention may therefore be used to provide benefits to the health and wellness of those who ingest the composition.

### SUMMARY

It is an objective of the present invention to provide a composition that is sufficiently safe to allow daily ingestion and that has an ability to promote the secretion of s-IgA into saliva in a continuous and age-independent manner. The above objective is achieved by the composition for promoting secretion of secretory IgA, comprising as an active ingredient Okinawa mozuku-derived fucoidan with a recommended daily intake of 1,000 mg or more. The composition has the mucosal immunostimulatory effect, anti-infection effect or anti-allergy effect and is expected to be used for ameliorating, mitigating, suppressing, treating or preventing diseases or symptoms on which these effects are expected to work, such as bacterial infection, viral infection, protozoan infection, food allergy, pollenosis, gastritis, enteritis, diarrhea, ulcerative colitis, rhinitis, bronchitis, bronchial asthma, Löffler's syndrome and stomatitis, as well as any symptoms resulting therefrom. The composition is also expected to be used in immunotherapies and induction of immunotolerance.

1. A composition for promoting secretion of secretory IgA, comprising as an active ingredient Okinawa mozuku-derived fucoidan with a recommended daily intake of 1,000 mg or more.

2. The composition according to claim 1, wherein the secretory IgA is secretory IgA in saliva.

3. A mucosal immunostimulatory composition, an anti-infection composition or an anti-allergy composition, comprising as an active ingredient Okinawa mozuku-derived fucoidan with a recommended daily intake of 1,000 mg or more.

4. The composition according to claim 1, wherein the composition is for ingestion by an individual having a rate of s-IgA secretion into saliva of 150 μg/min or less.

5. The composition according to claim 2, wherein the composition is for ingestion by an individual having a rate of s-IgA secretion into saliva of 150 μg/min or less.

6. The composition according to claim 3, wherein the composition is for ingestion by an individual having a rate of s-IgA secretion into saliva of 150 μg/min or less.
